(19) United States
(12) Patent Application Publication
(10) Pub. No.: US 2009/0130239 A1
(43) Pub. Date: May 21, 2009

(54) METHOD FOR PREVENTING OR TREATING EDEMA OR DERMATITIS USING GLEHNIA LITTORALIS FR. SCHMIDT ET MIQUEL EXTRACT

(75) Inventors: Ho Kyounng KIM, Daejeon (KR); Do Yeon Lee, Daejeon (KR); Byung-Kil Choo, Daejeon (KR); Myeong Sook Cheon, Daejeon (KR); Taesook Yoon, Seoul (KR)

Correspondence Address: MERCHANT & GOULD PC, P.O. BOX 2903, MINNEAPOLIS, MN, 55402-0903, US

(73) Assignee: Korea Institute of Oriental Medicine, Daejeon (KR)

(21) Appl. No.: 12/266,022

(22) Filed: Nov. 6, 2008

(30) Foreign Application Priority Data
Nov. 15, 2007 (KR) 10-2007-0116656

(51) Int. Cl. A61K 36/23; A61P 17/00; A61P 37/02

(52) U.S. Cl. 424/773

(57) ABSTRACT

The present invention relates to a method for preventing or treating edema or dermatitis using Glehnia littoralis Fr. Schmidt et Miquel extract. The Glehnia littoralis Fr. Schmidt et Miquel extract extracted using water, alcohol or a mixture thereof as a solvent inhibits production or secretion of cytokine regulators, the inflammatory mediators, but has no side effects such as toxicity, so that the extract can be effectively used for preventing and treating edema or different types of dermatitis accompanying inflammation.

This application claims benefit of Serial No. 10-2007-0116656, filed Nov. 15, 2007, in South Korea and which application is incorporated herein by reference. A claim of priority to all, to the extent appropriate is made.

### BACKGROUND OF THE INVENTION

1. Field of the Invention

The present invention relates to a method for preventing or treating edema or dermatitis using Glehnia littoralis Fr. Schmidt et Miquel extract.

2. Description of the Related Art

Inflammation is a sort of in vivo defensive reaction against tissue damage or against diverse stimuli or infectious agents from outside of body. Inflammation is a complicated series of pathological phenomenon comprising enzyme activation, inflammatory substance secretion, humoral/cellular infiltration, migration, tissue disturbance and hypeiplasia, for which various inflammatory mediators and immune cells in local blood vessel and body fluid are interacted. Inflammation reaction progresses as follows. First, macrophages are gathered to wound area to attack bacteria invaded. Then, blood plasma is accumulated on the wound area and blood flow increases. At last, external symptoms such as fever, erythema, edema and pain are generated. If inflammation reaction occurs continuously or excessively, it progresses to major pathological phenomena of disease (hypersensitivity, chronic inflammation), causing serious disorders.

Most frequently prescribed drugs for treating inflammation are non-steroidal anti-inflammatory drugs SAIDS). The non-steroidal anti-inflammatory drugs inhibit the enzyme activity of cyclooxygenase (COX) which is involved in biosynthesis of prostaglandin from arachidonic acid, resulting in anti-inflammatory effect. However, this kind of drugs causes side effects such as gastrointestinal dysfunction, hepatopathy and renal impairement, suggesting that long-term use is limited. Therefore, it is requested to develop a novel anti-inflammatory painkiller having excellent anti-inflammatory activity without side effects in order to be administered for a long term. Accordingly, recent studies have been focused on the development of an effective drug from natural resources.

Glehnia littoralis Fr. Schmidt et Miquel is a perennial herb belonging to Umbelliferae family, which is distributed in sandy soil near the coastline of northern pacific countries. In general, this plant is collected in summer and fall. Then, terrestrial stems are eliminated and roots and rhizomes are used as medicinal parts. It has long been known that Glehnia littoralis Fr. Schmidt et Miquel has excellent effect in underground parts. Particularly, its root makes the lung strong, indicating that it has effects on most respiratory diseases including pulmonary tuberculosis, pneumonia, bronchitis, sputum, cough, etc (Pan Zehui et al, Glehnia F. Schmidt ex Miquel, 3:61. 1867. Flora of China 14:173. 2005; Woon-gok-bon-cho-hak, 2004). Glehnia littoralis Fr. Schmidt et Miquel contains triterpenoid, stigmasterol, β-sitosterol, alkaloid and starch, and these components are effective in curing bronchial mucosa and increase secretions, leading to anti-tussive, mucolytic effect and excellent antipyretic effect (Chinese Pharmacopeia, 2005). However, there have been no studies or reports so far saying that Glehnia littoralis Fr. Schmidt et Miquel extract has a treatment effect on dermatitis.

Glehnia littoralis Fr. Schmidt et Miquel is in danger of extinction because of recent environmental changes and elimination of habitat by rapid industrial coastal development. So, it has been designated as an endangered or a protective plant by Ministry of Environment, Korea Forest Service and Culture Heritage Administration of Korea. Therefore, it is important to create continuous demand to save this plant from being extinct by proving the medicinal effect of this precious but loosing traditional cultural resource as a medicinal herb. It is also important to develop an applicable material therefrom to produce a value-added product from such a rare traditional medicinal herb, which resultantly contributes for securing excellent natural resources, improvement of national competitiveness and establishment of basis for the development of pro-environmental functional materials.

The present inventors completed this invention by confirming that Glehnia littoralis Fr. Schmidt et Miquel extract inhibits generation of cytokines, the inflammatory mediators, and also inhibits inflammation in an animal model with dermatitis induced by phorbol myristate acetate (TPA).

### SUMMARY OF THE INVENTION

It is an object of the present invention to provide a method for preventing or treating edema or dermatitis using Glehnia littoralis Fr. Schmidt et Miquel extract.

It is an object of the present invention to provide a method for preventing or treating edema or dermatitis using Glehnia littoralis Fr. Schmidt et Miquel extract.

The Glehnia littoralis Fr. Schmidt et Miquel extract of the present invention reduced the levels of serum cytokines such as interleukin-1β (IL-1β) and tumor necrosis factor-α (TNF-α) increased in an inflammation animal model with dermatitis induced by phorbol myristate acetate (TPA) to the levels of the control, resulting in the alleviation of epidermal hyperproliferation and reducing vascular permeability, indicating the inhibition of inflammation. Therefore, the Glehnia littoralis Fr. Schmidt et Miquel extract of the present invention can be effectively used for preventing or treating edema or various types of dermatitis including hyperergia, allergic dermatitis, contact dermatitis, atopic dermatitis, dermato-allergy and urticana.

### BRIEF DESCRIPTION OF THE DRAWINGS

The application of the preferred embodiments of the present invention is best understood with reference to the accompanying drawings, wherein:

FIG. 1 is a graph illustrating the inhibition of production of IL-1β, the inflammatory cytokine, in serum of the inflammation mouse model by Glehnia littoralis Fr. Schmidt et Miquel ethanol aqueous solution extract,

FIG. 1 is a graph illustrating the inhibition of production of TNF-α, the inflammatory cytokine, in serum of the inflammation mouse model by Glehnia littoralis Fr. Schmidt et Miquel ethanol aqueous solution extract,

FIG. 3 is a graph illustrating the reduction of thickness of edema induced in an ear of the inflammation mouse model by Glehnia littoralis Fr. Schmidt et Miquel ethanol aqueous solution extract,

FIG. 4 is a graph illustrating the decrease of weight of edema induced in an ear of the inflammation mouse model by Glehnia littoralis Fr. Schmidt et Miquel ethanol aqueous solution extract,

FIG. 5 is a graph illustrating the decrease of index of edema induced in an ear of the inflammation mouse model by Glehnia littoralis Fr. Schmidt et Miquel ethanol aqueous solution extract,

FIG. 6 is a diagram illustrating the inhibition of immune cell infiltration and epidermal hyperproliferation induced in ear tissues of the inflammation mouse model by Glehnia littoralis Fr. Schmidt et Miquel ethanol aqueous solution extract,

FIG. 7 is a graph illustrating the reduction of thickness of edema induced in an ear of the chronic inflammation mouse model by Glehnia littoralis Fr. Schmidt et Miquel ethanol aqueous solution extract,

FIG. 8 is a graph illustrating the decrease of index of edema induced in an ear of the chronic inflammation mouse model by Glehnia littoralis Fr. Schmidt et Miquel ethanol aqueous solution extract,

FIG. 9 is a graph illustrating the inhibition of vascular hyperpermeability induced by acetic acid by Glehnia littoralis Fr. Schmidt et Miquel ethanol aqueous solution extract.

### DESCRIPTION OF THE PREFERRED EMBODIMENTS

Hereinafter, the present invention is described in detail.

The Glehnia littoralis Fr. Schmidt et Miquel extract of the present invention is prepared by the method comprising the following steps:

1) extracting dried Glehnia littoralis Fr. Schmidt et Miquel using an extraction solvent;

2) cooling and filtering the extract of step 1); and

3) vacuum-concentrating the filtered extract of step 2) and drying thereof.

In the above method, the Glehnia littoralis Fr. Schmidt et Miquel of step 1) can be purchased or cultivated. Roots or rhizomes of Glehnia littoralis Fr. Schmidt et Miquel can be used but roots are preferred.

In the above method, the extraction solvent is water, alcohol or a mixture thereof. Herein, the alcohol is preferably C₁-C₄ lower alcohol and the lower alcohol is exemplified by ethanol or methanol. The preferable amount of the solvent is 5-15 times the amount of dried Glehnia littoralis Fr. Schmidt et Miquel, and more preferably 10 times the amount, but not always limited thereto. The extraction temperature is preferably 40˜80° C. and more preferably 50˜60° C., but not always limited thereto. The extraction time is preferably 2˜6 hours and more preferably 2 hours, but not always limited thereto. The extraction is preferably repeated 1-5 times and more preferably 3 times, but not always limited thereto.

In this method, the vacuum concentration of step 3) is preferably performed by using rotary vacuum evaporator, but not always limited thereto. The extract can be dried by hot air drying, vacuum drying or freeze drying, and freeze drying is preferred, but not always limited thereto.

The present invention provides a method for preventing or treating edema or dermatitis comprising administering a therapeutically effective dose of Glehnia littoralis Fr. Schmidt et Miquel extract to a subject. The dermatitis herein is exemplified by hyperergia, allergic dermatitis, contact dermatitis, atopic dermatitis, dermato-allergy or urticaria.

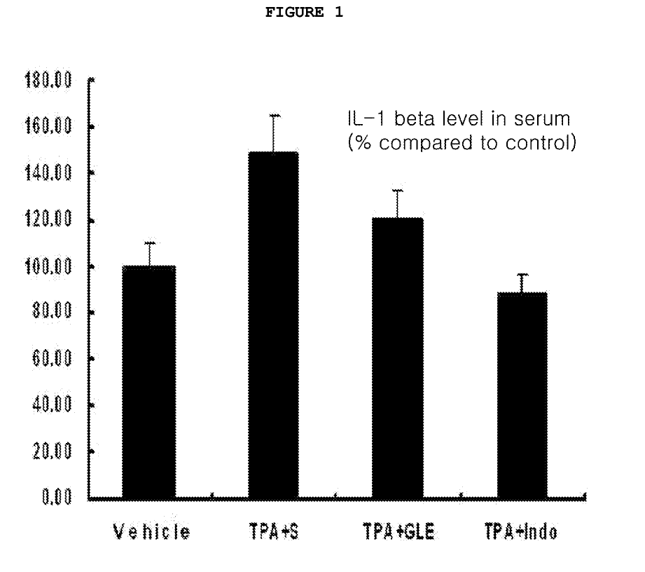

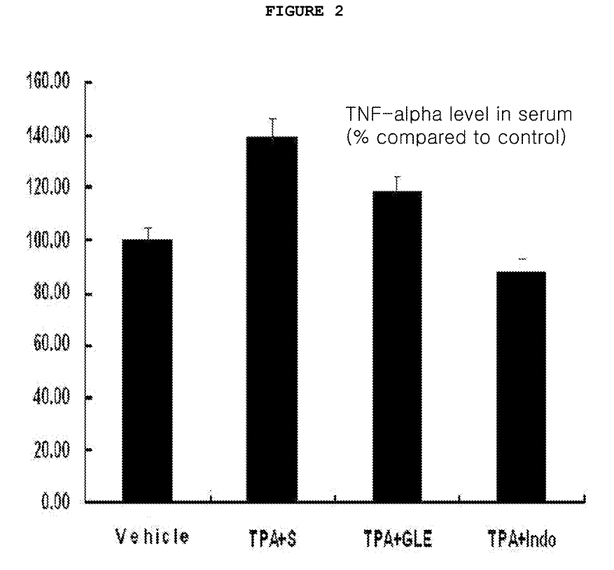

To investigate whether Glehnia littoralis Fr. Schmidt et Miquel extract could inhibit the expression of cytokine in serum, the Glehnia littoralis Fr. Schmidt et Miquel extract of the present invention was administered in mice and then the expression levels of cytokines such as IL-1β and TNF-α, the inflammatory mediators, in the mice serum were measured by ELISA. As a result, the treatment of the Glehnia littoralis Fr. Schmidt et Miquel extract of the present invention inhibited generations of IL-1β and TNF-α, confirming that the Glehnia littoralis Fr. Schmidt et Miquel extract of the present invention could inhibit inflammatory cytokines (see FIGS. 1 and 2).

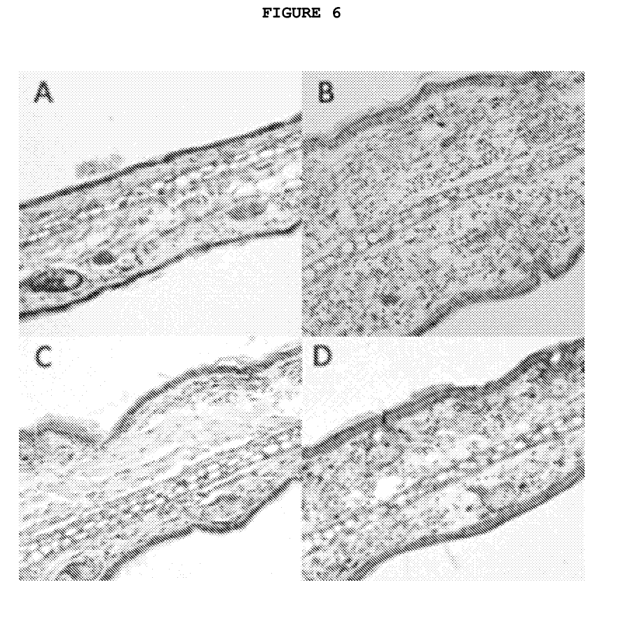

For histopathological analysis of anti-inflammation effect of Glehnia littoralis Fr. Schmidt et Miquel extract, a mouse with edema and dermatitis was sacrificed and ear tissues were extracted therefrom. The ear tissues were thin-sectioned by using cryocut microtome and the sections were stained with H&E, followed by observation. As a result, when Glehnia littoralis Fr. Schmidt et Miquel extract was administered, immune cell infiltration and epidermal hyperproliferation induced by TPA were inhibited, which was as significant as the inhibitory effect of indomethacin in the positive control (see FIG. 6).

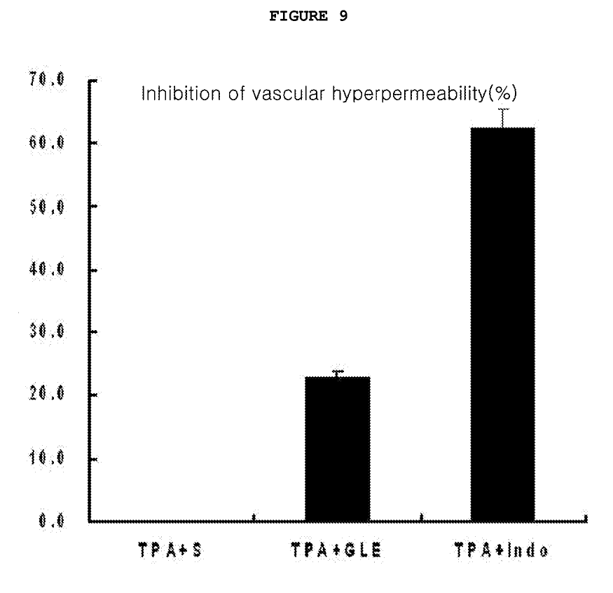

It was also investigated in this invention whether Glehnia littoralis Fr. Schmidt et Miquel extract could lower vascular permeability. Glehnia littoralis Fr. Schmidt et Miquel extract was administered orally to a mouse, followed by intravenous injection with Evans blue saline. Acetic acid was administered by intraperitoneal injection. The abdomen was opened and the inside of the abdominal cavity was washed with saline. Pigment content in the filtrate prepared by filtering the above solution with glass wool was measured with spectrometer. As a result, the Glehnia littoralis Fr. Schmidt et Miquel extract was confirmed to inhibit vascular permeability significantly (see FIG. 9).

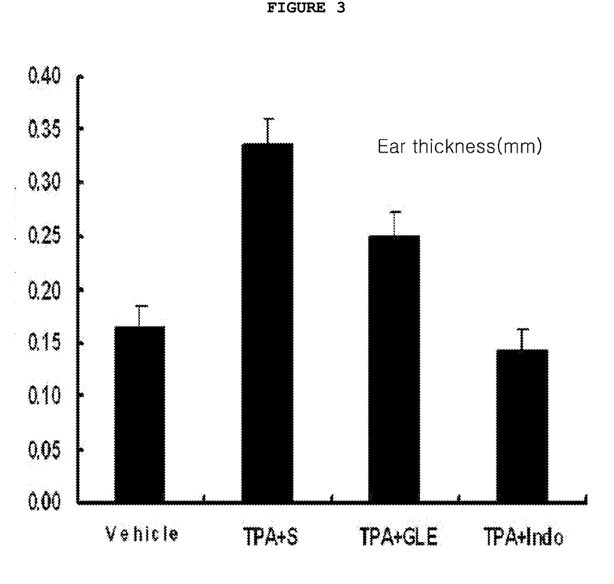

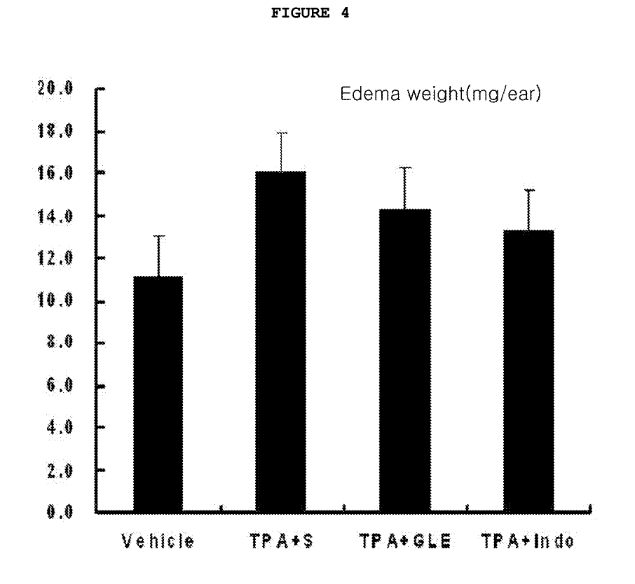

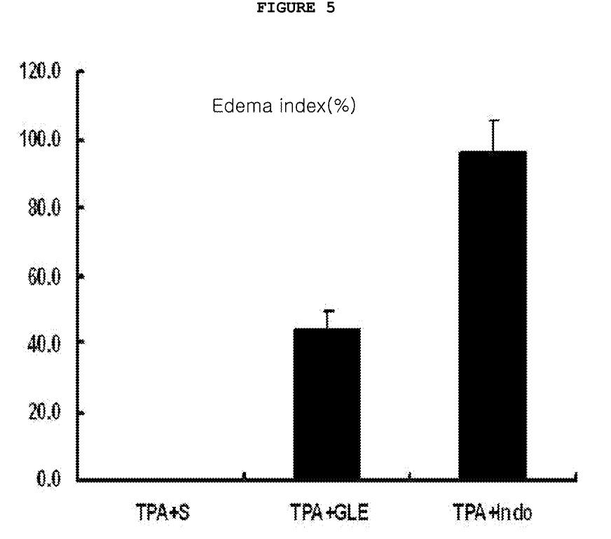

In this invention, inhibition effect of Glehnia littoralis Fr. Schmidt et Miquel extract on edema was also investigated. Mice with edema and dermatitis were prepared by local and repeated application of TPA (12-O-Tetrade-canoylphobol 13-acetate), to which Glehnia littoralis Fr. Schmidt et Miquel extract was administered. As a result, the treatment of the Glehnia littoralis Fr. Schmidt et Miquel extract of the present invention reduced the thickness and weight of edema and edema index, compared with the control, suggesting that the Glehnia littoralis Fr. Schmidt et Miquel extract of the present invention has excellent anti-inflammatory effect (see FIGS. 3, 4, 5, 7 and 8).

The composition of the present invention can include, in addition to the Glehnia littoralis Fr. Schmidt et Miquel extract, one or more effective ingredients having the same or similar function to the Glehnia littoralis Fr. Schmidt et Miquel extract.

The Glehnia littoralis Fr. Schmidt et Miquel extract can be administered orally or parenterally and be used in general forms of pharmaceutical formulation.

The Glehnia littoralis Fr. Schmidt et Miquel extract of the present invention can be prepared for oral or parenteral administration by mixing with generally used diluents or excipients such as fillers, extenders, binders, wetting agents, disintegrating agents and surfactant. Solid formulations for oral administration are tablets, pills, powders, granules and capsules. These solid formulations are prepared by mixing the Glehnia littoralis Fr. Schmidt et Miquel extract of the present invention with one or more suitable excipients such as starch, calcium carbonate, sucrose or lactose, gelatin, etc. Except for the simple excipients, lubricants, for example magnesium stearate, talc, etc, can be used. Liquid formulations for oral administrations are suspensions, solutions, emulsions and syrups, and the above-mentioned formulations can contain various excipients such as wetting agents, sweeteners, aromatics and preservatives in addition to generally used simple diluents such as water and liquid paraffin. Formulations for parenteral administration are sterilized aqueous solutions, water-insoluble excipients, suspensions, emulsions, lyophilized preparations and suppositories. Water insoluble excipients and suspensions can contain, in addition to the active compound or compounds, propylene glycol, polyethylene glycol, vegetable oil like olive oil, injectable ester like ethylolate, etc. Suppositories can contain, in addition to the active compound or compounds, witepsol, macrogol, tween 61, cacao butter, laurin butter, glycerogelatin, etc.

The effective dosage of the Glehnia littoralis Fr. Schmidt et Miquel extract of the present invention can be determined according to weight, age, gender, health condition, diet, administration frequency, administration method, excretion and severity of a disease. The dosage is 0.1˜100 mg/kg per day, preferably 30˜80 mg/kg per day, and more preferably 50˜60 mg/kg per day, and administration frequency is preferably 1˜6 times a day.

The Glehnia littoralis Fr. Schmidt et Miquel extract of the present invention can be administered alone or treated together with surgical operation, hormone therapy, chemotherapy and biological regulators.

The Glehnia littoralis Fr. Schmidt et Miquel extract of the present invention is evaluated to be a safe substance since its estimated LD₅₀ value is much greater than 2 g/kg in mice, which is confirmed by acute and repeated toxicity assay with mice tested via oral administration.

The present invention also provides a health food for the preventing or improving edema or dermatitis containing Glehnia littoralis Fr. Schmidt et Miquel extract as an active ingredient.

The Glehnia littoralis Fr. Schmidt et Miquel extract of the present invention can be used as a food additive. In that case, the Glehnia littoralis Fr. Schmidt et Miquel extract of the present invention can be added as it is or as mixed with other food components according to the conventional method. The mixing ratio of active ingredients can be regulated according to the purpose of use (prevention, health enhancement or treatment). In general, to produce health food or beverages, the Glehnia littoralis Fr. Schmidt et Miquel extract of the present invention is added preferably by up to 15 weight part and more preferably by up to 10 weight part. However, if long term administration is required for health and hygiene or regulating health condition, the content can be lower than the above but higher content can be accepted as well since the Glehnia littoralis Fr. Schmidt et Miquel extract of the present invention has been proved to be very safe.

The food herein is not limited. For example, the Glehnia littoralis Fr. Schmidt et Miquel extract of the present invention can be added to meats, sausages, breads, chocolates, candies, snacks, cookies, pizza, ramyuns, flour products, gums, dairy products including ice cream, soups, beverages, tea, drinks, alcohol drinks and vitamin complex, etc, and in wide sense, almost every food applicable in the production of health food can be included.

The composition for health beverages of the present invention can additionally include various flavors or natural carbohydrates, etc, like other beverages. The natural carbohydrates above can be one of monosaccharides such as glucose and fructose, disaccharides such as maltose and sucrose, polysaccharides such as dextrin and cyclodextrin, and glucose alcohols such as xylitol, sorbitol and erythritol. Besides, natural sweetening agents such as thaumatin and stevia extract, and synthetic sweetening agents such as saccharin and aspartame can be included as a sweetening agent. The content of the natural carbohydrate is preferably 0.01-0.04 g and more preferably 0.02-0.03 g in 100 Ml of the composition.

In addition to the ingredients mentioned above, the Glehnia littoralis Fr. Schmidt et Miquel extract of the present invention can include in a variety of nutrients, vitamins, minerals, flavors, coloring agents, pectic acid and its salts, alginic acid and its salts, organic acid, protective colloidal viscosifiers, pH regulators, stabilizers, antiseptics, glycerin, alcohols, carbonators which used to be added to soda, etc. The Glehnia littoralis Fr. Schmidt et Miquel extract of the present invention can also include natural fruit juice, fruit beverages and/or fruit flesh addable to vegetable beverages. All the mentioned ingredients can be added singly or together. The mixing ratio of those ingredients does not matter in fact, but in general, each can be added by 001-0.1 weight part per 100 weight part of the Glehnia littoralis Fr. Schmidt et Miquel extract of the present invention.

The present invention also provides a composition for preventing or improving edema or dermatitis containing Glehnia littoralis Fr. Schmidt et Miquel extract as an active ingredient as cosmetics.

The cosmetics containing the Glehnia littoralis Fr. Schmidt et Miquel extract of the present invention as an active ingredient can be formulated as general emulsified cosmetics and solubilized cosmetics. The emulsified cosmetics are exemplified by lotion, cream and essence, and the solubilized cosmetics are exemplified by skin.

The proper formulation of the cosmetics can be exemplified by solution, gel, solid or dough anhydride, emulsion prepared by dispersing oil phase on water phase, suspension, microemulsion, microcapsule, microgranule, ionic (liposome) or non-ionic vesicle, cream, skin, lotion powder, spray, and conceal stick. In addition, the cosmetics can be formulated as an aerosol composition containing a foam or compressed propellant.

The cosmetics of the present invention can include, in addition to the Glehnia littoralis Fr. Schmidt et Miquel extract of the present invention, a supplement generally used in the field of cosmetics such as fatty substance, organic solvent, resolvent, concentrate, gelling agent, softener, antioxidant, suspending agent, stabilizer, foaming agent, odorant, surfactant, water, ionic or non-ionic emulsifying agent, filler, sequestering agent, chelating agent, preserving agent, vitamin, blocker, moisturing agent, essential oil, dye, pigment, hydrophilic or hydrophobic activator, lipid vesicle or other components generally used in cosmetics.

Practical and presently preferred embodiments of the present invention are illustrative as shown in the following Examples, Experimental Examples and Manufacturing Examples.

However, it will be appreciated that those skilled in the art, on consideration of this disclosure, may make modifications and improvements within the spirit and scope of the present invention.

### Example 1

### Preparation of Glehnia Littoralis Fr. Schmidt et Miguel Extract

### <1-1> Preparation of Glehnia littoralis Fr. Schmidt et Miguel Ethanol Aqueous Solution Extract

The Glehnia littoralis Fr. Schmidt et Miquel used in this invention was collected in Geumhwajeong-ri Toseong-myeon, Goseong-gun, Gangwon-Do, Korea and dried. 100 g of pulverized roots of the Glehnia littoralis Fr. Schmidt et Miquel was added to 1 liter of 701% ethanol aqueous solution, followed by stirring. The mixture was heated, followed by reflux-extraction at 50-60° C. for 2 hours. The filtrate was separated. The herb extract was vacuum-concentrated at 50-55° C., followed by freeze-drying to give 11-12 g of powdered extract of the herb composition.

### <1-2> Preparation of Glehnia littoralis Fr. Schmidt et Miguel Water Extract

Extraction was performed by the same manner as described in Example <1-1> except that water was used instead of 701% ethanol aqueous solution as an extraction solvent. As a result, 7 g ˜8 g of extract was obtained.

### <1-3> Preparation of Glehnia littoralis Fr. Schmidt et Miguel Ethanol Extract

Extraction was performed by the same manner as described in Example <1-1> except that 100% ethanol was used instead of 70% ethanol aqueous solution as an extraction solvent. As a result, 12 g of extract was obtained.

### <1-4> Preparation of Glehnia littoralis Fr. Schmidt et Miguel Methanol Extract

Extraction was performed by the same manner as described in Example <1-1> except that 100% methanol was used instead of 700% ethanol aqueous solution as an extraction solvent. As a result, 11 g of extract was obtained.

### Experimental Example 1

### Anti-Inflammatory Effect of Glehnia littoralis Fr. Schmidt et Miguel Extract on TPA Induced Acute Dermatitis Animal Model

### <1-1> Preparation of Mouse Animal Model

C57BL/6J male mouse at 5 weeks (DAE HAN BIOLINK CO., LTD.), the most widely used inflammation animal model, was adapted to the experimental environment for 1 week. Total 40 mice were group into 4 (10 mice per each group). TPA (12-O-Tetrade-canoylphobol 13-acetate) was locally applied (2.5 ug/20 ul acetone) to the right ears of 6 week old mice to induce edema and dermatitis. The left ears of the mice were used as the negative control, to which an excipient (20 ul of acetone) was treated. The experimental group was treated with Glehnia littoralis Fr. Schmidt et Miquel extract (400 mg/kg) by intraperitoneal injection one hour before TPA treatment. The positive control group was administered with indomethacin (10 mg/kg) one hour before TPA treatment and the negative control was administered with saline by the same manner.

### <1-2> Cytokine Expression in Serum

In order to analyze the expression patterns of inflammatory cytokines such as IL-1β and TNF-α in immune system in serum of the mice respectively administered with Glehnia littoralis Fr. Schmidt et Miquel ethanol aqueous solution extract, water extract, ethanol extract and methanol extract, prepared in Example 1. ELISA (R&D) was performed with the serum prepared in Experimental Example 1. Every sample was stored at less than −20° C. until use.

TABLE 1

| | Inhibition of | Inhibition of |
|---|---|---|
| | IL-1β production | TNF-αproduction |
| Glehnia littoralis Fr. Schmidt | 41.95% | 46.70% |
| et Miquel ethanol aqueous solution | | |
| extract | | |
| Glehnia littoralis Fr. Schmidt | 38.50% | 42.25% |
| et Miquel water extract | | |
| Glehnia littoralis Fr. Schmidt | 40.25% | 44.55% |
| et Miquel ethanol extract | | |
| Glehnia littoralis Fr. Schmidt | 41.25% | 42.90% |
| et Miquel methanol extract | | |

As a result, all kinds of Glehnia littoralis Fr. Schmidt et Miquel extracts were confirmed to inhibit the productions of IL-1β and TNF-α. In particular, IL-1β production was inhibition by Glehnia littoralis Fr. Schmidt et Miquel ethanol aqueous solution extract by 41.95% and TNF-α production was inhibited by the same by 46.7%, suggesting that Glehnia littoralis Fr. Schmidt et Miquel ethanol aqueous solution extract demonstrated the strongest inflammatory cytokine inhibition effect (see FIGS. 1 and 2). So, Glehnia littoralis Fr. Schmidt et Miquel ethanol aqueous solution extract was used for the following experiments.

### <1-3> Analysis of Edema Index

Inflammation was induced for 6 hours as described in Experimental Example <1-1>, and then the animal was sacrificed. Disk fragment of 6 mm in diameter was obtained from each ear, followed by measurement of weight and thickness of edema. The thickness of edema taken from the experimental group mouse treated with TPA was 0.34 mm. When the mouse was treated with Glehnia littoralis extract (GLE), the thickness of edema was reduced to 0.25 mm, which was 49.7% reduction. The weight of edema of the experimental group mouse treated with TPA was also reduced from 16 mg to 15 mg by the treatment of Glehnia littoralis extract. Edema index was also decreased by the treatment of Glehnia littoralis extract (44.51%). The above results indicate that the Glehnia littoralis extract of the present invention has excellent anti-inflammatory activity in animal models (see FIGS. 3, 4 and 5).

### <1-4> Histopathological Analysis

Inflammation was induced for 6 hours by the same manner as described in Experimental Example <1-1> and then the animal was sacrificed. Ear tissues were extracted from the mouse and thin-sectioned by using cryocut microtome, followed by staining with H&E. As a result, pathologic growth phenomena caused by epidermal hyperproliferation and thickening were observed in skin tissues of the experimental group (B) mouse treated with TPA, compared with the control (A). In the experimental group mouse, edema and inflammation accompanying symptoms of migration and infiltration of polymorphonuclear leukocytes were induced. In the meantime, in the experimental group (C) administered with Glehnia littoralis Fr. Schmidt et Miquel extract, infiltration of immune cells and epidermal hyperproliferation were inhibited, compared with the TPA treated experimental group (B), which was as significant as the inhibition by indomethacin in the positive control (D) (see FIG. 6).

### Experimental Example 2

### Anti-Inflammatory Effect of Glehnia littoralis Fr. Schmidt et Miguel Extract on TPA Induced Chronic Dermatitis Animal Model

### <2-1> Preparation of Animal

C57BL/6J male mouse at 5 weeks (DAE HAN BIOLINK CO., LTD.), the most widely used inflammation animal model, was adapted to the experimental environment for 1 week. Total 40 mice were grouped into 4 (10 mice per each group). TPA (12-O-Tetrade-canoylphobol 13-acetate) was locally applied (2.5 ug/20 ul acetone) to the right ears of 6 week old mice to induce edema and dermatitis. The treatment of TPA was carried out one time in every other day, totally five times for inducing chronic animal model. The left ears of the mice were used as the negative control, to which an excipient (20 ul of acetone) was treated. The experimental group was treated with Glehnia littoralis Fr. Schmidt et Miquel extract (400 mg/kg) by intraperitoneal injection for 10 days totally. The positive control group was administered with indomethacin (10 mg/kg) and the negative control was administered with saline by the same manner.

### <2-2> Analysis of Edema Index

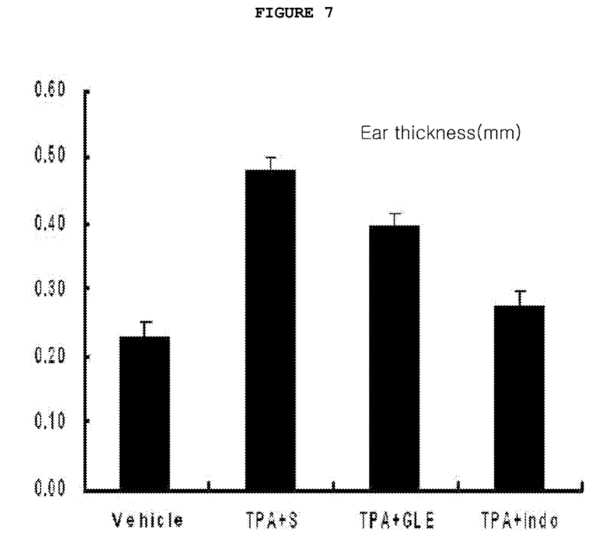

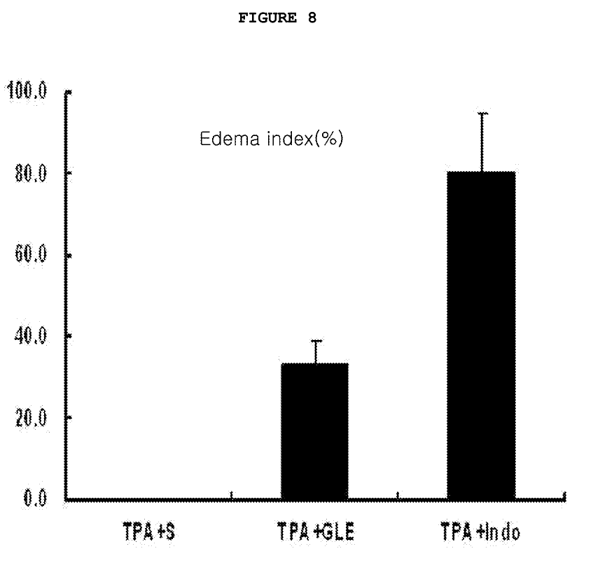

Inflammation was induced for 10 days as described in Experimental Example <2-1>, and then the animal was sacrificed. Disk fragment of 6 mm in diameter was obtained from each ear, followed by measurement of thickness and index of edema. The thickness of edema taken from the experimental group mouse treated with TPA was 0.48 mm. When the mouse was treated with Glehnia littoralis extract, the thickness of edema was reduced to 0.39 mm, which was 33.46% reduction. Edema index was also decreased by the treatment of Glehnia littoralis extract (33%). The above results indicate that the Glehnia littoralis extract of the present invention has excellent anti-inflammatory activity in chronic inflammation animal model (see FIGS. 7 and 8).

### Experimental Example 3

### Inhibition of Vascular Hyperpermeability Induced by Acetic Acid

30 ICR male mice (weight: 19˜21 g) were randomly grouped into three (10 mice per group). The mice were orally administered with Glehnia littoralis Fr. Schmidt et Miquel extract (400 mg/kg). 20 minutes later, 2.5% Evans blue saline was injected into tail vein of the mice at the concentration of 0.1 ml/10 g. 20 minutes after the intravenous injection, 0.6% acetic acid (0.1 m/10 g) was administered by intraperitoneal injection. 20 minutes later, the abdomen was opened and the inside of the abdominal cavity was washed with saline. Pigment content in the filtrate prepared by filtering the above solution with glass wool was measured with spectrometer at 590 nm. As a result, the Glehnia littoralis Fr. Schmidt et Miquel extract demonstrated significant inhibition effect on vascular hyperpermeability (approximately 23%) (see FIG. 9).

### Experimental Example 4

### Toxicity Test of Glehnia littoralis Fr. Schmidt et Miguel Extract

Acute toxicity test and repeated toxicity test were performed in mice as follows to confirm the stability of Glehnia littoralis Fr. Schmidt et Miquel extract. It is generally understood in acute toxicity test that when an animal does not show any abnormal signs or symptoms or death at the dose of 1 g/kg, the target compound is regarded as a safe substance. Based on that, the Glehnia littoralis Fr. Schmidt et Miquel extract of the present invention was confirmed to be safe.

As for repeated toxicity test, 30 C57BL6/J male mice were grouped into three (10 mice per group), to which Glehnia littoralis Fr. Schmidt et Miquel extract was administered for 4 weeks at different concentrations of 500 mg/kg, 1000 mg/kg, and 2000 mg/kg every day. The control group was administered with water instead. Signs of toxicity were investigated by observing weight changes, serum biochemical assay and histological assay for 4 weeks. As a result, death was not observed even at maximum concentration of 2000 mg/kg of Glehnia littoralis Fr. Schmidt et Miquel extract and any abnormal symptoms such as weight gain or loss, changes in feed consumption, decrease of mobility, lack of motion, dyspnea and hair loss were not observed, either. No abnormal signs were found from blood test and biopsy. Therefore, the Glehnia littoralis Fr. Schmidt et Miquel extract of the present invention was confirmed to be a safe substance. Any abnormal weight changes were not observed by naked eye. Biochemical test with serum confirmed that no abnormal changes in levels of AST, ALT, glucose and cholesterol were observed. As a result of biopsy, no abnormal signs in the liver, kidney, spleen and thymus were observed.

The Manufacturing Examples of the composition for the present invention are described hereinafter.

### Manufacturing Example 1

### Preparation of Pharmaceutical Formulations

Pharmaceutical formulations containing the Glehnia littoralis Fr. Schmidt et Miquel extract of the present invention were prepared as follows.

<1-1>Preparation of powders

| | |
|---|---|
| Extract of Example <1-1> | 2 g |
| Lactose | 1 g |

Powders were prepared by mixing all the above components, which were filled in airtight packs according to the conventional method for preparing powders.

### <1-2> Preparation of Tablets

| | |
|---|---|
| Extract of Example <1-1> | 100 mg |
| Corn starch | 100 mg |
| Lactose | 100 mg |
| Magnesium stearate | 2 mg |

Tablets were prepared by mixing all the above components by the conventional method for preparing tablets.

### <1-3> Preparation of Capsules

| | |
|---|---|
| Extract of Example <1-1> | 100 mg |
| Corn starch | 100 mg |
| Lactose | 100 mg |
| Magnesium stearate | 2 mg |

Capsules were prepared by mixing all the above components, which were filled in gelatin capsules according to the conventional method for preparing capsules.

### <14> Preparation of Pills

| | |
|---|---|
| Extract of Example <1-1> | 1 g |
| Lactose | 1.5 g |
| Glycerin | 1 g |
| Xylitol | 0.5 g |

Pills were prepared by mixing all the above components according to the conventional method for preparing pills. Each pill contained 4 g of the mixture.

### <1-5> Preparation of Granules

| | |
|---|---|
| Extract of Example <1-1> | 150 mg |
| Soybean extract | 50 mg |
| Glucose | 200 mg |
| Starch | 600 mg |

All the above components were mixed, to which 100 mg of 30% ethanol was added. The mixture was dried at 60° C. and the prepared granules were filled in packs.

### Manufacturing Example 2

### Preparation of Foods

Foods containing the Glehnia littoralis Fr. Schmidt et Miquel extract of the present invention were prepared as follows.

### <2-1> Preparation of Flour Food

0.5˜5.0 weight % of the extract of Example <1-1> was added to the flour. Health enhancing foods such as bread, cake, cookies, crackers and noodles were prepared with the flour mixture according to the conventional method.

### <2-2> Preparation of Dairy Products

5˜10 weight % of the extract of Example <1-1> was added to milk. Health enhancing dairy products such as butter and ice cream were prepared with the milk mixture according to the conventional method.

### <2-3> Preparation of Sun-Sik

Brown rice, barley, glutinous rice and Yulmu (Job's tears) were gelatinized according to the conventional method, dried and pulverized to obtain 60-mesh powders.

Black soybean, black sesame and wild sesame were steamed and dried according to the conventional method and pulverized to obtain 60-mesh powders.

The extract of Example <1-1> was concentrated under reduced pressure, spray-dried and pulverized to obtain 60-mesh dry powders.

Sun-Sik was prepared by mixing the dry powders of the grains, seeds and the extract of Example <1-2> according to the below ratio.

Grains (brown rice: 30 weight %, Yulmu: 15 weight %, barley: 20 weight %),

Seeds (wild sesame: 7 weight %, black soybean: 8 weight %, black sesame: 7 weight %),

Dry powders of the extract of Example <1-1> (3 weight %),

Ganoderma lucidum (0.5 weight %),

Rehmannia glutinosa (0.5 weight %)

### Manufacturing Example 3

### Preparation of Beverages

### <3-1> Preparation of Health Beverages

| | | |
|---|---|---|
| Extract of Example <1-1> | 1000 | mg |
| Citric acid | 1000 | mg |
| Oligosaccharide | 100 | g |
| Maesil (Prunus mume) Extract | 2 | g |
| Taurine | 1 | g |
| Purified water | up to 900 | Ml |

The above constituents were mixed according to the conventional method for preparing health beverages. The mixture was heated at 85° C. for 1 hour with stirring and then filtered. The filtrate was loaded in 2 liter sterilized containers, which were sealed and sterilized again, stored in a refrigerator until they would be used for the preparation of a composition for health beverages.

The constituents appropriate for favorite beverages were mixed according to the preferred mixing ratio but the composition ratio can be adjusted according to regional and national preferences, etc.

### <3-2> Preparation of Vegetable Juice

Health enhancing vegetable juice was prepared by adding 5 g of the extract of Example <1-1> of the present invention to 1,000 Ml of tomato or carrot juice according to the conventional method.

### <3-3> Preparation of Fruit Juice

Health enhancing vegetable juice was prepared by adding 1 g of the extract of Example <1-1> of the present invention to 1,000 Ml of apple or grape juice according to the conventional method.

### Manufacturing Example 4

### Preparation of Cosmetics

Functional cosmetics containing the Glehnia littoralis Fr. Schmidt et Miquel extract of the present invention as an active ingredient can be prepared. The present inventors prepared functional cosmetics, which are exemplified by such emulsified cosmetics as lotion, cream and essence; and such solubilized cosmetics as skin, etc.

### <4-1> Preparation of Emulsified Cosmetics

Emulsified cosmetics were prepared according to the composition shown in Table 1. The method for the preparation is as follows.

1) heating the mixture comprising the raw materials 1-9 at 65-70° C.;

2) adding the raw material 10 to the mixture of step 1);

3) dissolving the mixture comprising the raw materials 11-13 by heating at 65-70° C.;

4) slowly adding the mixture of step 2) during performing step 3), followed by emulsification at 8,000 rpm for 2-3 minutes;

5) dissolving the raw material 14 in water and then adding the solution to the mixture of step 4), followed by emulsification for 2 minutes;

6) weighing the raw materials 15-17, which were added to the mixture of step 5), followed by emulsification for 30 seconds; and

7) degassing the mixture of step 6) finished with the emulsification process and then cooling thereof at 25-35° C. to give emulsified cosmetics.

TABLE 2
Compositions of emulsified formulations 1, 2 and 3

| | | | | Emulsified |
|---|---|---|---|---|
| | | Emulsified | Emulsified | formu- |
| | | formulation | formulation | lation |
| Composition | | 1 | 2 | 3 |
| 1 | Stearic acid | 0.3 | 0.3 | 0.3 |
| 2 | Stearyl alcohol | 0.2 | 0.2 | 0.2 |
| 3 | Glyceryl monostearate | 1.2 | 1.2 | 1.2 |
| 4 | Wax | 0.4 | 0.4 | 0.4 |
| 5 | Polyoxyethylenesorbitan | 2.2 | 2.2 | 2.2 |
| | monolauric acid ester | | | |
| 6 | Paraoxybenzoic acid methyl | 0.1 | 0.1 | 0.1 |
| 7 | Paraoxybenzoic acid propyl | 0.05 | 0.05 | 0.05 |
| 8 | Cetyl ethyl hexanoate | 5 | 5 | 5 |
| 9 | Triglyceride | 2 | 2 | 2 |
| 10 | Cyclomethicone | 3 | 3 | 3 |
| 11 | Distilled water | to 100 | to 100 | to 100 |
| 12 | Concentrated glycerin | 5 | 5 | 5 |
| 13 | Triethanolamine | 0.15 | 0.15 | 0.15 |
| 14 | Polyacrylic acid polymer | 0.12 | 0.12 | 0.12 |
| 15 | Pigment | 0.0001 | 0.0001 | 0.0001 |
| 16 | Flavor | 0.10 | 0.10 | 0.10 |
| 17 | Extract of Example <1-1> | 0.0001 | 1 | 10 |

### <4-2> Preparation of Solubilized Cosmetics

Solubilized cosmetics were prepared according to the composition shown in Table 2. The method for the preparation is as follows.

1) adding the raw materials 2-6 to the raw material 1 (purified water), which were dissolved by using Agi-mixer;

2) adding the raw materials 8-11 to the raw material 7 (alcohol) and completely dissolved; and

3) slowly adding the mixture of step 2) to the mixture of step 1), followed by solubilization.

TABLE 3
Compositions of solubilized formulations 1, 2 and 3

| | | | | solubilized |
|---|---|---|---|---|
| | | solubilized | solubilized | formu- |
| | | formulation | formulation | lation |
| Composition | | 1 | 2 | 3 |
| 1 | Purified water | to 100 | to 100 | to 100 |
| 2 | Concentrated glycerin | 3 | 3 | 3 |
| 3 | 1,3-butyleneglycol | 2 | 2 | 2 |
| 4 | EDTA-2Na | 0.01 | 0.01 | 0.01 |
| 5 | Pigment | 0.0001 | 0.0002 | 0.0002 |
| 6 | Extract of Example <1-1> | 0.1 | 5 | 5 |
| 7 | Alcohol (95%) | 8 | 8 | 8 |
| 8 | Paraoxybenzoic acid methyl | 0.1 | 0.1 | 0.1 |
| 9 | Polyoxyethylene | 0.3 | 0.3 | 0.3 |
| | hydrogenated ester | | | |
| 10 | Flavor | 0.15 | 0.15 | 0.15 |
| 11 | Cyclomethicone | — | — | 0.2 |

Those skilled in the art will appreciate that the conceptions and specific embodiments disclosed in the foregoing description may be readily utilized as a basis for modifying or designing other embodiments for carrying out the same purposes of the present invention. Those skilled in the art will also appreciate that such equivalent embodiments do not depart from the spirit and scope of the invention as set forth in the appended Claims.

What is claimed is:

1. A method for preventing or treating dermatitis comprising administering a therapeutically effective dose of Glehnia littoralis Fr. Schmidt et Miquel extract to a subject in need thereof.

2. The method according to claim 1, wherein the Glehnia littoralis Fr. Schmidt et Miquel extract is extracted by using water, C₁-C₄ lower alcohol or a mixture thereof as a solvent.

3. The method according to claim 2, wherein the C₁-C₄ lower alcohol is ethanol or methanol.

4. The method according to claim 1, wherein the Glehnia littoralis Fr. Schmidt et Miquel extract inhibits production of interleukin-1β(IL-1β) in serum.

5. The method according to claim 1, wherein the Glehnia littoralis Fr. Schmidt et Miquel extract inhibits production of tumor necrosis factor-α(TNF-α) in serum.

6. The method according to claim 1, wherein the Glehnia littoralis Fr. Schmidt et Miquel extract inhibits vascular hyperpermeability.

7. The method according to claim 1, wherein the dermatitis is selected from the group consisting of hyperergia, allergic dermatitis, contact dermatitis, atopic dermatitis, dermato-allergy and urticaria.

8. A method for preventing or treating edema comprising administering a therapeutically effective dose of Glehnia littoralis Fr. Schmidt et Miquel extract to a subject in need thereof.
